(19) United States
(12) Patent Application Publication
(10) Pub. No.: US 2024/0069324 A1
(43) Pub. Date: Feb. 29, 2024

(54) LENS UNIT, IMAGE PICKUP APPARATUS, AND ENDOSCOPE

(71) Applicant: OLYMPUS CORPORATION, Tokyo (JP)

(72) Inventor: Mitsuru HAGIHARA, Kamiina-gun (JP)

(73) Assignee: OLYMPUS CORPORATION, Tokyo (JP)

(21) Appl. No.: 18/387,174

(22) Filed: Nov. 6, 2023

Related U.S. Application Data
(63) Continuation of application No. PCT/JP2021/027022, filed on Jul. 19, 2021.

(51) Int. Cl. G02B 23/24; G02B 3/00

(52) U.S. Cl. CPC G02B 23/243; G02B 3/0068

(57) ABSTRACT

A lens unit includes: a first optical device including a first glass substrate including a first principal surface and a second principal surface, and a resin lens disposed on the second principal surface; a second optical device including a second glass substrate including a third principal surface and a fourth principal surface, in which the third principal surface is disposed facing the second principal surface, and a substantially rectangular aperture layer made of metal that is disposed on the third principal surface and that has four corner regions cut out; and an adhesive layer that adhesively bonds the first optical device and the second optical device, in which four corner regions do not sandwich the aperture layer between the first optical device and the second optical device.

### CROSS REFERENCE TO RELATED APPLICATION

This application is a continuation application of PCT/JP2021/027022 filed on Jul. 19, 2021, the entire contents of which are incorporated herein by this reference.

### BACKGROUND OF THE INVENTION

### 1. Field of the Invention

The present invention relates to a lens unit including a hybrid lens device in which a resin lens is disposed on a glass substrate, an image pickup apparatus including the lens unit having the hybrid lens device, and an endoscope including the image pickup apparatus including the lens unit having the hybrid lens device.

### 2. Description of the Related Art

It is important to reduce a diameter of a lens unit of an image pickup apparatus disposed in a distal end portion of an endoscope for alleviating invasiveness.

International Publication No. 2017/203592 discloses a lens unit that is a wafer-level stacked body that allows efficient manufacturing of the lens unit with a small diameter. The wafer-level stacked body is manufactured by cutting a stacked wafer in which a plurality of lens wafers, each including a plurality of lens devices, are stacked so as to sandwich adhesive layers.

### SUMMARY OF THE INVENTION

A lens unit of an embodiment includes: a first optical device including a first glass substrate including a first principal surface and a second principal surface on a side opposite to the first principal surface, and a resin lens disposed on the second principal surface; a second optical device including a second glass substrate including a third principal surface and a fourth principal surface on a side opposite to the third principal surface, in which the third principal surface is disposed facing the second principal surface, and a substantially rectangular aperture layer made of metal that is disposed on the third principal surface and that has a shape of a cross-section orthogonal to an optical axis with four corner regions cut out; and an adhesive layer that adhesively bonds the first optical device and the second optical device, in which at least any one of four corner regions does not sandwich the aperture layer between the first optical device and the second optical device.

An image pickup apparatus of an embodiment includes: a lens unit and an image pickup unit that receives light of an optical image condensed by the lens unit, in which the lens unit includes: a first optical device including a first glass substrate including a first principal surface and a second principal surface on a side opposite to the first principal surface, and a resin lens disposed on the second principal surface; a second optical device including a second glass substrate including a third principal surface and a fourth principal surface on a side opposite to the third principal surface, in which the third principal surface is disposed facing the second principal surface, and a substantially rectangular aperture layer made of metal that is disposed on the third principal surface and that has a shape of a cross-section orthogonal to an optical axis with four corner regions cut out; and an adhesive layer that adhesively bonds the first optical device and the second optical device, in which at least any one of four corner regions does not sandwich the aperture layer between the first optical device and the second optical device.

An endoscope of an embodiment includes an image pickup apparatus including a lens unit and an image pickup unit that receives light of an optical image condensed by the lens unit, in which the lens unit includes: a first optical device including a first glass substrate including a first principal surface and a second principal surface on a side opposite to the first principal surface, and a resin lens disposed on the second principal surface; a second optical device including a second glass substrate including a third principal surface and a fourth principal surface on a side opposite to the third principal surface, in which the third principal surface is disposed facing the second principal surface, and a substantially rectangular aperture layer made of metal that is disposed on the third principal surface and that has a shape of a cross-section orthogonal to an optical axis with four corner regions cut out; and an adhesive layer that adhesively bonds the first optical device and the second optical device, in which at least any one of four corner regions does not sandwich the aperture layer between the first optical device and the second optical device.

### BRIEF DESCRIPTION OF THE DRAWINGS

FIG. 1 is a perspective view of an image pickup apparatus of a first embodiment;

FIG. 2 is a cross-sectional view taken along line II-II of FIG. 1:

FIG. 3 is a cross-sectional view taken along line III-III of FIG. 1;

FIG. 4 is a cross-sectional view of a lens unit of the first embodiment including an aperture layer;

FIG. 5 is an exploded cross-sectional view for explaining a manufacturing method of the image pickup apparatus of the first embodiment;

FIG. 6 is a cross-sectional view for explaining the manufacturing method of the image pickup apparatus of the first embodiment;

FIG. 7 is a cross-sectional view for explaining a manufacturing method of the lens unit of the first embodiment including an aperture layer:

FIG. 8A is a cross-sectional view of a lens unit of a modification 1 of the first embodiment including an aperture layer;

FIG. 8B is a cross-sectional view of a lens unit of a modification 2 of the first embodiment including an aperture layer;

FIG. 9 is a perspective view of an image pickup apparatus of a second embodiment;

FIG. 10 is a cross-sectional view taken along line X-X of FIG. 9:

FIG. 11A is a cross-sectional view of a lens unit of the second embodiment including an aperture layer;

FIG. 11B is a cross-sectional view of a lens unit of a modification of the second embodiment including an aperture layer, and

FIG. 12 is a perspective view of an endoscope of a third embodiment.

### DETAILED DESCRIPTION OF THE PREFERRED EMBODIMENTS

### First Embodiment

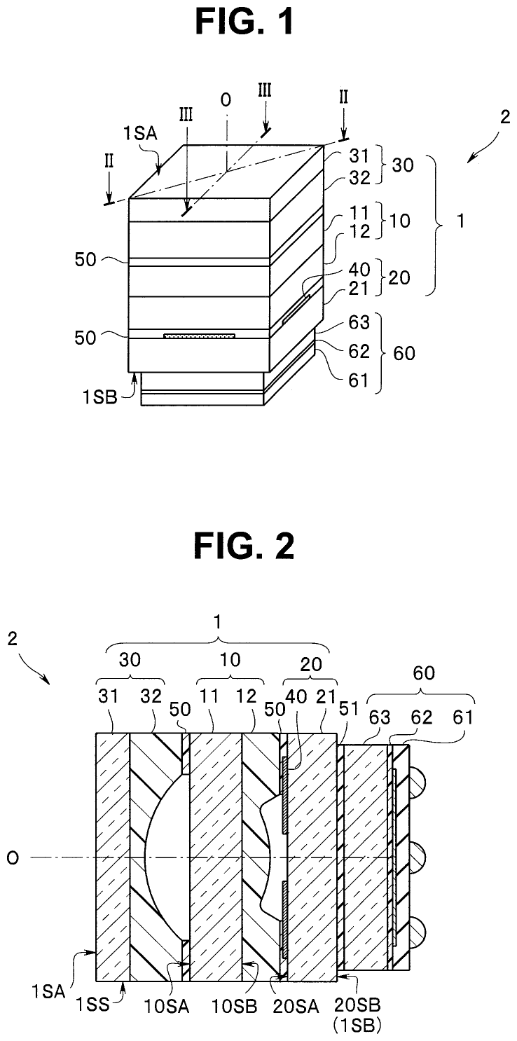

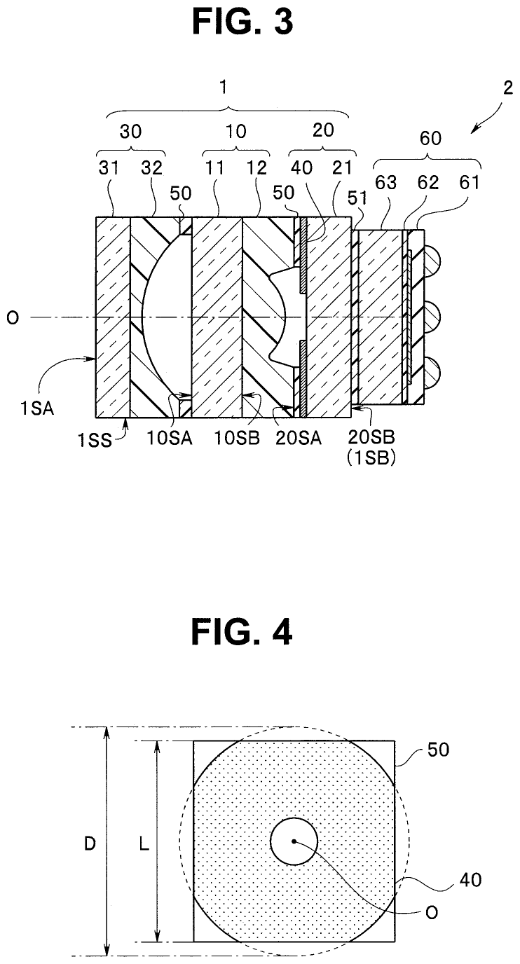

As shown in FIG. 1 to FIG. 3, an image pickup apparatus 2 of an embodiment includes a lens unit 1 and an image pickup unit 60 of the embodiment. A reference numeral O indicates an optical axis of the lens unit 1. The image pickup unit 60 receives light of an optical image condensed by the lens unit 1 to convert the optical image into an image pickup signal.

Note that in the following description, the drawings based on the embodiments are schematic illustrations. The relation between the thickness and the width of each portion, the ratio in thickness and the relative angle of each portion, and the like differ from the actual components. There are also some portions with different dimensional relations and ratios among the drawings. Illustration of part of the constituent elements will be omitted.

The lens unit 1 includes an incident surface 1SA in substantially a square with one side D and an emission surface 1SB on a side opposite to the incident surface 1SA. The lens unit 1 includes a third optical device 30 including the incident surface 1SA, a first optical device 10, and a second optical device 20 including the emission surface 1SB. The third optical device 30, the first optical device 10, and the second optical device 20 are stacked in this order, and sizes of the principal surfaces of the third optical device 30, the first optical device 10, and the second optical device 20 are substantially the same.

The first optical device 10 has, as a base body, a first glass substrate 11 including a first principal surface 10SA and a second principal surface 10SB on a side opposite to the first principal surface 10SA. The first optical device 10 is a hybrid lens device including a convex lens 12 made of resin on the second principal surface 10SB.

The second optical device 20 has, as a base body, a second glass substrate 21 including a third principal surface 20SA and a fourth principal surface 20SB on a side opposite to the third principal surface 20SA. The third principal surface 20SA is disposed facing the second principal surface 10SB. The fourth principal surface 20SB is the emission surface 1SB of the lens unit 1. The second optical device 20 includes an aperture layer 40 made of metal that is disposed on the third principal surface 20SA of the second glass substrate 21. The second glass substrate 21 may be a glass filter that removes unnecessary infrared light (for example, light with a wavelength equal to or greater than 700 nm).

The third optical device 30 is a hybrid lens device having a third glass substrate 31 as a base body and including a resin lens as a concave lens on a principal surface on a side opposite to the incident surface 1SA.

The first glass substrate 11, the second glass substrate 21, and the third glass substrate 31 are made of, for example, borosilicate glass, quartz glass, or sapphire glass.

The third optical device 30 and the first optical device 10, and the first optical device 10 and the second optical device 20 are respectively adhesively bonded by means of an adhesive layer 50 made of resin.

Note that a configuration of the lens unit of the present invention is not limited to the configuration of the lens unit 1, and is determined in accordance with the specification. For example, the lens unit may include a spacer element that defines a distance between the lenses and a plurality of aperture layers in addition to the lens device.

The image pickup unit 60 is adhesively bonded to the fourth principal surface 20SB (emission surface 1SB) of the second optical device 20 by means of an adhesive layer 51. In the image pickup unit 60, a cover glass 63 is adhesively bonded to an image pickup device 61 by means of an adhesive layer 62. The lens unit 1 forms a subject image on the image pickup device 61. The image pickup device 61 is a CMOS (complementary metal oxide semiconductor) light receiving element or a CCD (charge coupled device).

As shown in FIG. 4, a shape of the aperture layer 40 is substantially rectangular (square with four corners on an outer edge cut out). As will be described later, this is because a circular metal pattern with an outer diameter D is cut into a square with a width L by four cut lines. An opening at the center of the aperture layer 40 is an optical path.

The adhesive layer 50 is disposed in a cutout region of the aperture layer 40. In other words, four corner regions on the outer edge of the adhesive layer 50 with a rectangular shape of a cross-section orthogonal to the optical axis do not sandwich the aperture layer 40 between the first optical device 10 and the second optical device 20.

As already described, the adhesion of the adhesive layer 50 made of resin with the aperture layer 40 made of metal is not high. In contrast, the adhesion of the adhesive layer 50 with the resin lens 12 and the second glass substrate 21 is high. Further, in a region where the adhesive layer 50 does not sandwich the aperture layer 40 between the resin lens 12 and the second glass substrate 21, the residual stress is reduced.

In the adhesive layer 50 of the lens unit 1, the four corner regions that are most likely to be detached do not sandwich the aperture layer 40. Therefore, the lens unit 1 is easy to manufacture and is highly reliable.

<Manufacturing Method>

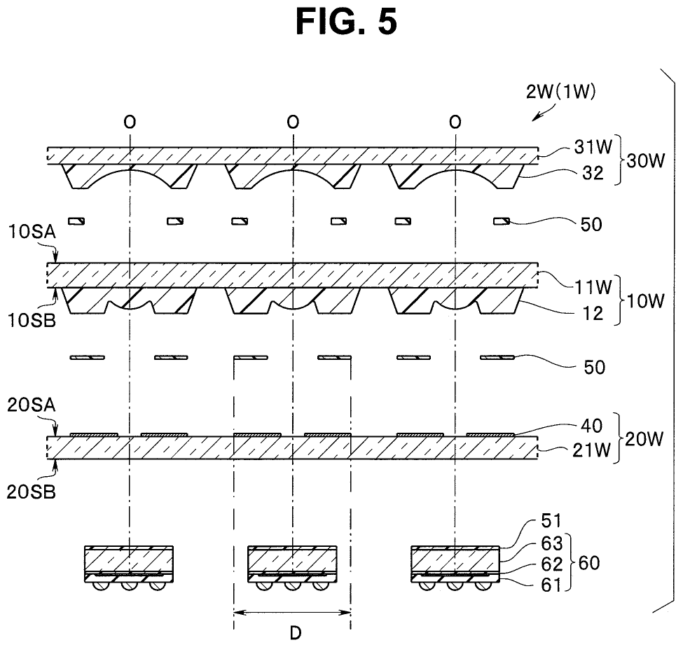

As shown in FIG. 5, the lens unit 1 is a wafer-level lens unit in a substantially rectangular parallelepiped shape that is manufactured by cutting a stacked wafer 1W in which a plurality of lens wafers respectively including a plurality of optical devices disposed in a matrix are stacked. Note that hereinafter, a manufacturing method of the image pickup apparatus 2 by cutting a stacked wafer 2W in which the plurality of image pickup units 60 are disposed on the stacked wafer 1W will be described as an example.

A device wafer 10W including the plurality of first optical devices 10 is produced such that the resin lens 12 is disposed on a glass wafer 11W. It is preferable that energy curable resin should be used for the resin of the resin lens 12.

Cross-linking reaction or polymerization reaction of the energy curable resin proceeds by reception of energy such as heat, ultraviolet light, and electron beam from outside. For example, the energy curable resin includes transparent ultraviolet curing silicone resin, epoxy resin, or acrylic resin. Note that “transparent” means that a material has less light absorption and less scattering in such a degree that the material can endure in use in a use wavelength range.

The resin lens 12 is produced using a mold method in which uncured resin, which is thus liquid or gel, is disposed on the glass wafer 11W and ultraviolet light is irradiated to cure the resin in a state of being pressed by a mold having a recessed portion with a predetermined inner surface shape. Note that silane coupling treatment or the like is preferably performed on the glass wafer before the resin is disposed to improve an interface adhesive strength between the glass and the resin. A device wafer 30W is produced using the same method as the method for the device wafer 10W.

Since the inner surface shape of the mold is transferred to an outer surface shape of the resin lens manufactured using the mold method, it is possible to easily produce a configuration having an outer periphery portion which also functions as a spacer and an aspherical lens.

For example, a metal layer disposed on the third principal surface 20SA of a glass wafer 21W is patterned using a sputtering method so that a device wafer 20W including the plurality of aperture layers 40 is produced. The aperture layer 40 includes chromium or titanium as a main component. The “main component” means accounting for 90% or more by weight.

The plurality of aperture layers 40 patterned using a metal mask may be disposed on the glass wafer 21W. As shown in FIG. 7, the aperture layer 40 in the device wafer 20W is circular with the outer diameter D and includes the opening as the optical path at the center. The outer diameter D of the aperture layer 40 is designed so as to be greater than a distance of cut lines CL, that is, the width L of the incident surface 1SA of the lens unit 1.

The adhesive layer 50 is disposed on each of resin lenses 32 of the device wafer 30W and the resin lenses 12 of the device wafer 10W using a transfer method. The adhesive layer 50 may be disposed using an ink-jet method. The adhesive layer 50 is, for example, a thermosetting epoxy resin. The adhesive layer 50 may be, for example, a light-shielding layer including light shielding particles. The stacked wafer 1W is produced such that the device wafer 30W, the device wafer 10W, and the device wafer 20W are stacked and adhesively bonded together.

The stacked wafer 2W is produced by adhesively bonding the plurality of image pickup units 60 to the stacked wafer 1W using the adhesive layer 51. The image pickup unit 60 is manufactured by cutting an image pickup wafer in which a glass wafer that becomes the cover glass 63 is adhesively bonded to a device wafer including the plurality of image pickup devices 61 using a transparent adhesive layer 62. The stacked wafer 2W may be produced by adhesively bonding the image pickup wafer to the stacked wafer 1W.

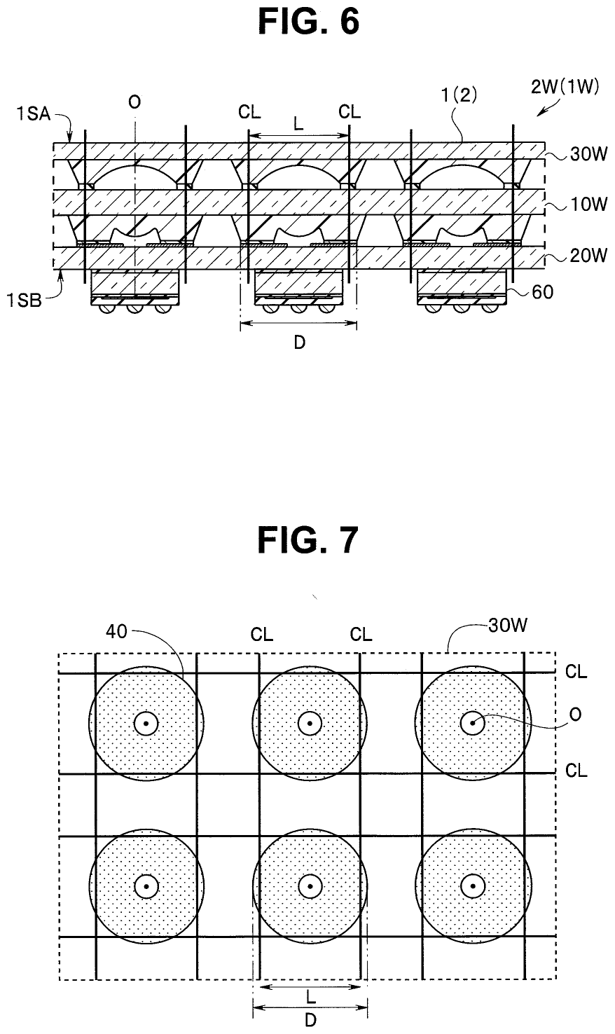

As shown in FIG. 6, the stacked wafer 2W is cut along the cut lines CL in a grid pattern so as to be made into individual pieces of the plurality of image pickup apparatuses 2 (lens unit 1). As shown in FIG. 7, the aperture layer 40 having a circular shape of a cross-section in a direction orthogonal to the optical axis is cut into substantially a circle having the outer shape with four straight lines, that is, a rectangle in such a shape that four corners are cut out in an arc, by the four cut lines CL.

In the adhesive layer 50 of the lens unit 1, the four corner regions that are most likely to be detached do not sandwich the aperture layer 40. Therefore, the lens unit 1 is easy to manufacture and is highly reliable.

The image pickup apparatus 2 may be produced such that the image pickup unit 60 is disposed on the lens unit 1 manufactured by cutting the stacked wafer 1W.

### Modifications of First Embodiment

Since lens units of embodiments and modifications described below are similar to and have the same functions as the functions of the lens unit 1, the components having the same functions will be assigned the same reference numerals, and the description will be omitted.

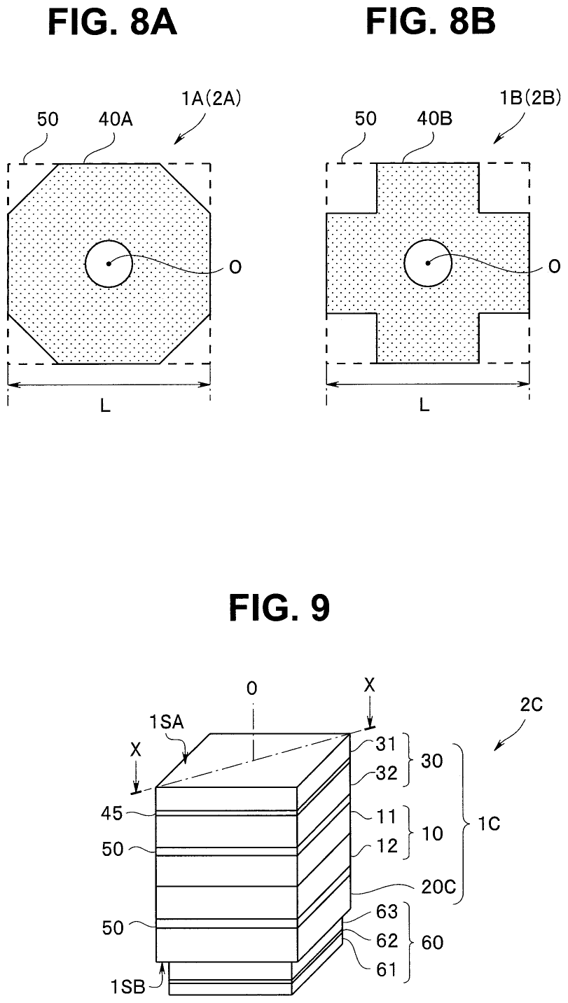

An aperture layer 40A of a lens unit 1A of a modification 1 shown in FIG. 8A and an aperture layer 40B of a lens unit 1B of a modification 2 shown in FIG. 8B both have four corner regions cut out.

Therefore, in the adhesive layer 50 that adhesively bonds the first optical device 10 and the second optical device 20 together, the four corner regions do not sandwich the aperture layer 40A. 40B between the first optical device 10 and the second optical device 20.

An image pickup apparatus 2A including the lens unit 1A and an image pickup apparatus 2B including the lens unit 1B have the same effects as the effects of the image pickup apparatus 2.

Note that in the lens unit 1, 1A, 1B, the four corner regions of the rectangular adhesive layer 50 do not sandwich the aperture layer 40 between the first optical device 10 and the second optical device 20. However, it goes without saying that even in the lens unit in which only one corner region of the adhesive layer 50 does not sandwich the aperture layer 40 between the first optical device 10 and the second optical device 20, manufacturing is easier and the reliability is higher as compared to the lens unit in which all the corner regions of the adhesive layer 50 sandwich the aperture layer 40 between the first optical device 10 and the second optical device 20. In other words, in the adhesive layer 50 of the embodiment, it is only necessary that at least any one of the four corner regions does not sandwich the aperture layer 40 between the first optical device 10 and the second optical device 20.

### Second Embodiment

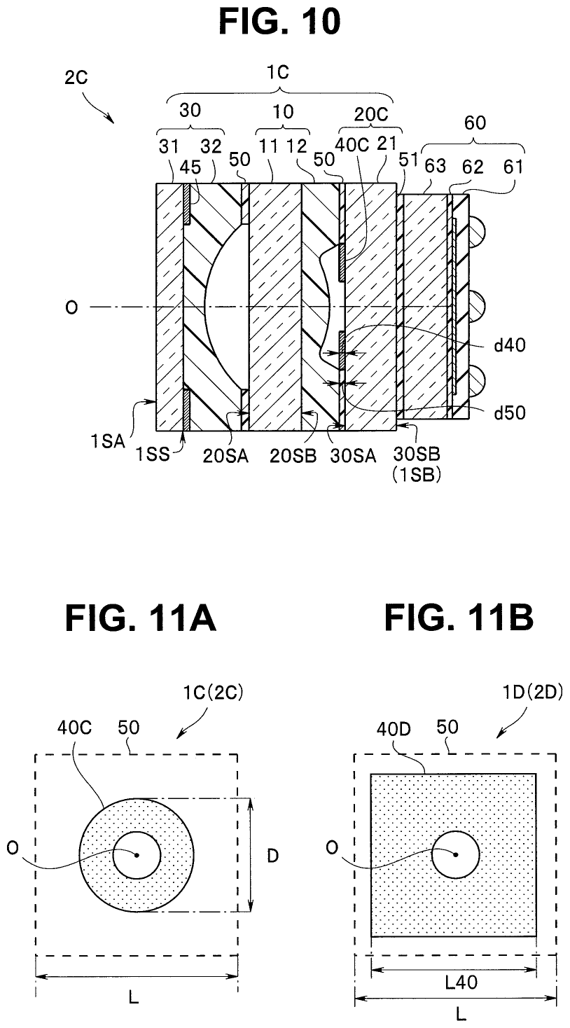

As shown in FIG. 9 and FIG. 10, an aperture layer 40C of an image pickup apparatus 2C of the present embodiment is not exposed on a side surface of a lens unit 1C. As shown in FIG. 11A, an outer diameter D of the aperture layer 40C is smaller than the width L of the lens unit 1C. Further, the adhesive layer 50 and the aperture layer 40C do not overlap in an optical axis direction. Note that a thickness d40 of the aperture layer 40C is substantially equal to a thickness d50 of the adhesive layer 50.

In the lens unit 1C, the adhesive layer 50 does not sandwich the aperture layer 40 between the first optical device 10 and the second optical device 20. Therefore, the lens unit 1C is easier to manufacture and is more highly reliable as compared to the lens unit 1.

The lens unit 1C includes a second aperture layer 45 made of metal between a third glass substrate 31 and the resin lens 32. An adhesion strength between the second aperture layer 45 and the resin lens 32 is higher than an adhesion strength between the second aperture layer 45 and the adhesive layer 50. Therefore, there is no problem when a corner region of the resin lens 32 contacts the second aperture layer 45. It goes without saying that also in the second aperture layer 45, the corner region may not be exposed on the side surface of the lens unit, similarly to the aperture layer 40.

An outer shape of an aperture layer 40D of a lens unit 1D of a modification shown in FIG. 11B is a square. A width L40 of the aperture layer 40D is smaller than the width L of the lens unit 1D. Since the adhesive layer 50 does not sandwich the aperture layer 40D between the first optical device 10 and the second optical device 20, an image pickup apparatus 2D including the lens unit 1D has the same effects as the effects of the image pickup apparatus 2C including the lens unit 1C.

Note that as long as the aperture layer 40 that overlaps the adhesive layer 50 is not exposed on the side surface of the lens unit 1, the lens unit may be in substantially a prism or a column having the side surfaces with chamfered corners.

### Third Embodiment

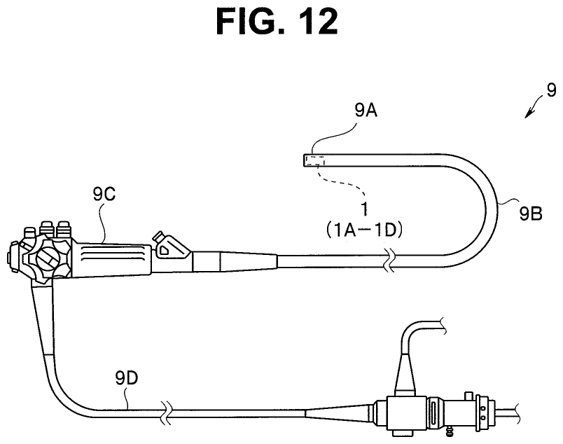

As shown in FIG. 12, an endoscope 9 of the present embodiment includes a distal end portion 9A, an insertion portion 9B extending from the distal end portion 9A, an operation portion 9C disposed on a proximal end side of the insertion portion 9B, and a universal cord 9D extending from the operation portion 9C.

The image pickup apparatus 2 (2A-2D) including the lens unit 1 (1A-1D) is disposed in the distal end portion 9A. An image pickup signal outputted from the image pickup apparatus 2 is transmitted to a processor (not shown) via a cable that passes through the universal cord 9D. A drive signal from the processor to the image pickup apparatus 2 is also transmitted via the cable that passes through the universal cord 9D.

As described, the lens unit 1 (1A-1D) is easy to manufacture and is highly reliable. Therefore, the endoscope 9 is easy to manufacture and is highly reliable.

The present invention is not limited to the aforementioned embodiments and the like, and various changes, combinations, and applications are available within the scope without departing from the gist of the invention.

What is claimed is:

1. A lens unit comprising:
a first optical device including: a first glass substrate including a first principal surface and a second principal surface on a side opposite to the first principal surface, and a resin lens disposed on the second principal surface;
a second optical device including: a second glass substrate including a third principal surface and a fourth principal surface on a side opposite to the third principal surface, wherein the third principal surface is disposed facing the second principal surface; and a substantially rectangular aperture layer made of metal that is disposed on the third principal surface and that has a shape of a cross-section orthogonal to an optical axis with four corner regions cut out; and
an adhesive layer that adhesively bonds the first optical device and the second optical device, wherein four corner regions do not sandwich the aperture layer between the first optical device and the second optical device.

2. The lens unit according to claim 1, wherein the adhesive layer and the aperture layer do not overlap in an optical axis direction.

3. The lens unit according to claim 2, wherein a thickness of the adhesive layer is substantially equal to a thickness of the aperture layer.

4. The lens unit according to claim 1, wherein the adhesive layer has a substantially rectangular shape of the cross-section with four corners cut out in an arc.

5. The lens unit according to claim 1, wherein the aperture layer includes chromium or titanium as a main component.

6. An image pickup apparatus comprising a lens unit and an image pickup unit that receives light of an optical image condensed by the lens unit, wherein
the lens unit comprises:
a first optical device including: a first glass substrate including a first principal surface and a second principal surface on a side opposite to the first principal surface; and a resin lens disposed on the second principal surface;
a second optical device including: a second glass substrate including a third principal surface and a fourth principal surface on a side opposite to the third principal surface, wherein the third principal surface is disposed facing the second principal surface; and a substantially rectangular aperture layer made of metal that is disposed on the third principal surface and that has a shape of a cross-section orthogonal to an optical axis with four corner regions cut out; and
an adhesive layer that adhesively bonds the first optical device and the second optical device, wherein four corner regions do not sandwich the aperture layer between the first optical device and the second optical device.

7. An endoscope comprising an image pickup apparatus comprising a lens unit and an image pickup unit that receives light of an optical image condensed by the lens unit, wherein
the lens unit comprises:
a first optical device including: a first glass substrate including a first principal surface and a second principal surface on a side opposite to the first principal surface; and a resin lens disposed on the second principal surface;
a second optical device including: a second glass substrate including a third principal surface and a fourth principal surface on a side opposite to the third principal surface, wherein the third principal surface is disposed facing the second principal surface; and a substantially rectangular aperture layer made of metal that is disposed on the third principal surface and that has a shape of a cross-section orthogonal to an optical axis with four corner regions cut out; and
an adhesive layer that adhesively bonds the first optical device and the second optical device, wherein four corner regions do not sandwich the aperture layer between the first optical device and the second optical device.
